(19) United States
(12) Patent Application Publication
(10) Pub. No.: US 2025/0138109 A1
(43) Pub. Date: May 1, 2025

(54) VERIFICATION DEVICE AND METHOD OF OPERATING VERIFICATION DEVICE

(71) Applicant: SAMSUNG ELECTRONICS CO., LTD., Suwon-si (KR)

(72) Inventors: CHISUNG BAE, Suwon-si (KR); Nahm il KOO, Suwon-si (KR)

(73) Assignee: SAMSUNG ELECTRONICS CO., LTD., Suwon-si (KR)

(21) Appl. No.: 18/923,190

(22) Filed: Oct. 22, 2024

(30) Foreign Application Priority Data
Nov. 1, 2023 (KR) 10-2023-0149032

(51) Int. Cl. G01R 31/66; C12M 1/34; H01L 27/092

(52) U.S. Cl. CPC G01R 31/66; C12M 41/46; H10D 84/85

(57) ABSTRACT

A verification device and a method of operating the verification device. The verification device includes: an electrode array including a plurality of electrodes configured to contact a culture medium for culturing a plurality of cells, and a plurality of analog front ends (AFEs) corresponding to the plurality of electrodes; and a control circuit including a plurality of first connection verifying circuits (CVCs) corresponding to the plurality of electrodes, wherein the control circuit is configured to verify at least one connection from among a plurality of first connections between the plurality of cells and the plurality of electrodes, and a plurality of second connections between the plurality of electrodes based on a result of verifying the plurality of first CVCs.

### CROSS-REFERENCE TO RELATED APPLICATION

This application is based on and claims priority under 35 U.S.C. § 119 to Korean Patent Application No. 10-2023-0149032, filed on Nov. 1, 2023, in the Korean Intellectual Property Office, the disclosure of which is incorporated by reference herein in its entirety.

### BACKGROUND

### 1. Field

The disclosure relates to a verification device of a microelectrode array and a method of operating the verification device.

### 2. Description of Related Art

A human biosignal may be analyzed using various methods. For example, signals from neurons measured by a plurality of electrodes may be used to analyze brain waves. In addition, a signal transfer mode of a brain may be interpreted by providing a current having a certain magnitude to neurons in order to observe intracellular signals. A microelectrode array (MEA) may include a plurality of electrodes, and an electrical signal generated at cellular level may be measured through each electrode from among the plurality of electrodes.

### SUMMARY

In accordance with an aspect of the disclosure, a verification device includes: an electrode array including a plurality of electrodes configured to contact a culture medium for culturing a plurality of cells, and a plurality of analog front ends (AFEs) corresponding to the plurality of electrodes; and a control circuit including a plurality of first connection verifying circuits (CVCs) corresponding to the plurality of electrodes, wherein the control circuit is configured to verify at least one connection from among a plurality of first connections between the plurality of cells and the plurality of electrodes, and a plurality of second connections between the plurality of electrodes based on a result of verifying the plurality of first CVCs.

Each first CVC from among the plurality of first CVCs may include: a first p-channel metal-oxide-semiconductor (PMOS) transistor connected to a first adjacent electrode that is adjacent to a target electrode connected to a target cell from among the plurality of electrodes, wherein the first PMOS transistor is configured to operate as a current source for the first adjacent electrode; and a first n-channel metal-oxide-semiconductor (NMOS) transistor connected to an input end of an adjacent AFE connected to a second adjacent electrode that is adjacent to the target electrode, wherein the first NMOS transistor is configured to form an electrical signal path with the first PMOS transistor.

The control circuit may be further configured to verify a first connection between the target cell and the second adjacent electrode based on a gain of the adjacent AFE connected to the second adjacent electrode.

The control circuit may be further configured to verify a first connection between a cell from among the plurality of cells and an electrode from among the plurality of electrodes by determining whether a gain of an AFE from among the plurality of AFEs corresponding to a first CVC from among the plurality of first CVCs has a first value.

The control circuit may be further configured to determine a degree of capacitive coupling of the adjacent AFE corresponding to the second adjacent electrode by sweeping a reference signal applied to a gate terminal of the first PMOS transistor of each first CVC from among the plurality of first CVCs.

The control circuit may be further configured to verify a second connection from among the plurality of second connections by determining whether the electrical signal path is formed between the first adjacent electrode, the second adjacent electrode, and the target electrode.

The control circuit may be further configured to verify the second connection by determining whether an impedance path is formed by a first impedance corresponding to the first adjacent electrode, a second impedance corresponding to the target electrode, and a third impedance corresponding to the second adjacent electrode.

The plurality of AFEs may include a direct-conversion AFE.

The first NMOS transistor may include a diode-connected NMOS transistor.

Each AFE of the plurality of AFEs may include a first p-channel metal oxide semiconductor (PMOS) transistor configured to operate as a current source, and the control circuit may be further configured to verify the plurality of second connections by determining whether an electrical signal path is formed between first PMOS transistors included in AFEs connected to the plurality of first CVCs.

The control circuit may further include a second CVC including a second p-channel metal oxide semiconductor (PMOS) transistor configured to operate as a current source for the culture medium, and a second n-channel metal oxide semiconductor (NMOS) transistor configured to form a current path with the second PMOS transistor.

The control circuit may be further configured to verify a connection between the plurality of AFEs and the plurality of electrodes by determining whether an electrical signal path is formed in each first CVC from among the plurality of first CVCs by activating the second PMOS transistor and sequentially activating first NMOS transistors included in the plurality of first CVCs.

The control circuit may be further configured to verify a connection between a target AFE and the adjacent AFE by determining whether an electrical signal path is formed by activating the first PMOS transistor of a first CVC connected to the target AFE from among the plurality of AFEs, and activating the first NMOS transistor of a first CVC connected to the adjacent AFE.

The control circuit may be further configured to verify an operation between the adjacent AFE and the first PMOS transistor by directly connecting the first PMOS transistor to the first NMOS transistor without the culture medium.

The control circuit may further include a selection logic circuit configured to activate a first target CVC connected to at least one target cell corresponding to a verification target from among the plurality of first CVCs.

The control circuit may be further configured to calculate a distribution of the at least one target cell by activating the first target CVC using the selection logic circuit.

The control circuit may be further configured to detect a small cell that is smaller than the target cell by performing image reconstruction based on a magnitude of a voltage applied between electrodes adjacent to the target cell being changed as a current path changes while a current is applied through the current source due to a cell membrane of the target cell having an impedance greater than an impedance of a surrounding cell.

The verification device may include a drug screening device.

In accordance with an aspect of the disclosure, a method of operating a verification device includes: selecting a plurality of target cells corresponding to a verification target according to a selection signal of a user; activating a plurality of first target connection verifying circuits (CVCs) connected to a plurality of target electrodes corresponding to the plurality of target cells; and verifying at least one connection from among a plurality of first connections between the plurality of target cells and the plurality of target electrodes, and a plurality of second connections between the plurality of target electrodes and a plurality of adjacent electrodes that are adjacent to the plurality of target electrodes.

In accordance with an aspect of the disclosure, a non-transitory computer-readable storage medium stores instructions which, when executed by a processor of a verification device, cause the verification device to: select a plurality of target cells corresponding to a verification target according to a selection signal of a user; activate a plurality of first target connection verifying circuits (CVCs) connected to a plurality of target electrodes corresponding to the plurality of target cells; and verify at least one connection from among a plurality of first connections between the plurality of target cells and the plurality of target electrodes, and a plurality of second connections between the plurality of target electrodes and a plurality of adjacent electrodes that are adjacent to the plurality of target electrodes.

Additional aspects of example embodiments will be set forth in part in the description which follows and, in part, will be apparent from the description, or may be learned by practice of the disclosure.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a diagram illustrating situations that may occur at an interface between electrodes of a microelectrode array (MEA), according to an embodiment;

FIG. 2 is a block diagram illustrating a verification device according to an embodiment;

FIG. 3 is a diagram illustrating a configuration of a verification device according to an embodiment;

FIG. 4 is a diagram illustrating a structure of a first connection verifying circuit (CVC) according to an embodiment;

FIG. 5 is a diagram illustrating a method of verifying a first connection between an electrode of an MEA and a cell according to an embodiment;

FIG. 6 is a diagram illustrating a method of verifying a second connection between electrodes of an MEA according to an embodiment;

FIG. 7 is a diagram illustrating a method of performing cross verification between a distribution of a target cell calculated by a control circuit and a cell image according to an embodiment;

FIG. 8 is a diagram illustrating a method of determining the presence of a small cell that is smaller than a target cell by a control circuit according to an embodiment;

FIG. 9 is a diagram illustrating a method of verifying integrity of a single channel of a first CVC according to an embodiment;

FIG. 10A is a diagram illustrating a simulation circuit for verifying connection between a culture medium and an electrode of an MEA according to an embodiment;

FIG. 10B is a diagram illustrating an amount of current applied to the simulation circuit of FIG. 10A that is input to each CVC through electrodes;

FIG. 10C is a diagram illustrating a difference between amplitudes of signals according to the amount of current for each electrode of FIG. 10B;

FIG. 11A is a diagram illustrating a simulation circuit for verifying connection between electrodes of an MEA according to an embodiment;

FIG. 11B is a diagram illustrating an amount of current applied to the simulation circuit of FIG. 11A that is input to each CVC through a cell and a cell membrane connected to the electrode;

FIG. 11C is a diagram illustrating a difference between amplitudes of signals according to the amount of current for each electrode of FIG. 11B; and

FIG. 12 is a flowchart illustrating a method of operating a verification device according to an embodiment.

### DETAILED DESCRIPTION

The following detailed structural or functional description is provided as an example only and various alterations and modifications may be made to the examples. Accordingly, the described embodiments are not intended to limit the disclosure, and the disclosure should be understood to include all changes, equivalents, and replacements.

Although terms such as first, second, and the like are used to describe various components, the components are not limited to such terms. Instead, these terms should be understood only to distinguish one component from another component. For example, a first component may be referred to as a second component, or similarly, the second component may be referred to as the first component.

It should be noted that if one component is described as being “connected”, “coupled”, or “joined” to another component, this may include an arrangement in which a third component may be “connected”, “coupled”, and “joined” between the first and second components, and also an arrangement in which the first component may be directly connected, coupled, or joined to the second component.

The singular forms “a”, “an”, and “the” are intended to include the plural forms as well, unless the context clearly indicates otherwise. It will be further understood that the terms “comprises/including” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms, including technical and scientific terms, used herein have the same meaning as commonly understood by one of ordinary skill in the art to which this disclosure pertains. Terms, such as those defined in commonly used dictionaries, should be construed to have meanings matching with contextual meanings in the relevant art, and are not to be construed to have an ideal or excessively formal meaning unless otherwise defined herein.

Hereinafter, embodiments will be described in detail with reference to the accompanying drawings. In the description below, like reference numerals refer to like components and a repeated description related thereto will be omitted.

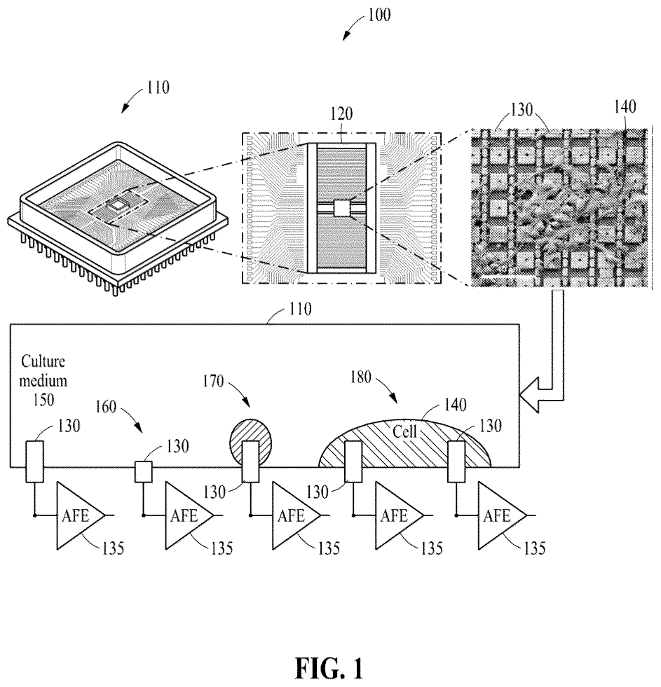

FIG. 1 is a diagram illustrating a microelectrode array (MEA) according to an embodiment, and various interface conditions that may occur at an interface between electrodes of the MEA. In particular, FIG. 1 shows a verification device 100 including an MEA 120 according to an embodiment, and various interface conditions, for example interface condition 160, interface condition 170, and interface condition 180, that may occur between interfaces of electrodes 130 of the MEA 120. According to embodiments, the MEA 120 may also be simply referred to as an “electrode array”.

The MEA 120 may include the electrodes 130 manufactured on a silicon chip, and cells 140 may be cultured or mounted on the electrodes 130 to measure electrical signals generated at a cellular level.

In some embodiments, the MEA 120 may include tens of thousands of channels within a chip, for example, to analyze a connection between neurons or brain cells at a network level. The MEA 120 may include the electrodes 130 arranged as a 64×64 array. The MEA 120 may include microelectrodes having a relatively small pitch, for example, 20 micrometers (um)×20 um, but embodiments are not limited thereto.

The electrodes 130 may be used for stimulation and sensing of the cells 140 to be cultured in a cell culture vessel 110. In some embodiments, hundreds to thousands of the electrodes 130 may be present on the silicon chip, which may allow an electrical signal to be measured at a cellular level.

The verification device 100 according to an embodiment may measure a state of the cells based on a contact impedance that occurs between the electrodes 130 and the cells 140.

The MEA 120 may mount the cell culture vessel 110 on a top surface of a package. The cell culture vessel 110 may store a culture medium 150 for culturing the cells 140. The cell culture vessel 110 may be placed on top of a chip including the MEA 120. The cell culture vessel 110 may include, for example, an inner ring having a space for placing and culturing the cells 140, and an outer ring having a space for storing the culture medium 150. The cells 140 may be, for example, neurons corresponding to a brain, or other types of neurons, however embodiments are not limited thereto.

The culture medium 150 may refer to a nutrient source, for example at least one of a liquid and gel which may be used to proliferate microorganisms or cells. The culture medium 150 may be referred to as a “medium” or a “culture liquid”. The culture medium 150 may be contained in a cell culture chamber or the cell culture vessel 110. The culture medium 150 may be, for example, a saline solution, but embodiments are not limited thereto.

In embodiments, the measurement of electrical signals at the cellular level as described above may be carried out through a number of operations as described below. First, a passivation layer of a chip may be peeled off to fabricate an electrode in the form of at least one of a planar hole and a pillar using etching, patterning, and deposition, and special packaging for cell culture may be performed. The process described may be performed using an eco-friendly or bio-friendly material, which may refer to a material which has little or no damaging effect on the environment or on biological material.

Because the process described above may be difficult to perform, an electrode 130 may be formed improperly, as shown in interface condition 160. In addition, in the process of culturing the cells 140 after forming an electrode 130, various abnormal situations may occur, such as the formation of a disturbing factor such as bubble which may disturb a connection between an electrode 130 and a cell, as shown in interface condition 170. Another abnormal situation may occur in which the same signal is detected from all of various electrodes due to a single cell 140 covering a plurality of electrodes 130, as shown in interface condition 180.

The verification device 100 according to an embodiment may determine an electrical connection between channels of the MEA 120 through the contact impedance, as well as an electrical connection between an analog front end (AFE) 135, examples of which are described below, and the culture medium 150 including the cells 140, and between the electrodes 130 attached to the AFEs 135, thereby allowing any problems or issues to be detected in various locations, including the electrode, the packaging, and the cell culture.

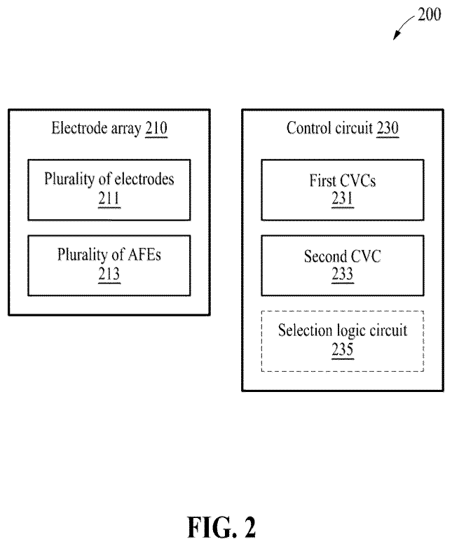

FIG. 2 is a block diagram illustrating a verification device according to an embodiment. Referring to FIG. 2, a verification device 200 according to an embodiment includes an electrode array 210 and a control circuit 230. In embodiments, the verification device 200 may correspond to the verification device 100 discussed above, but embodiments are not limited thereto.

The electrode array 210 may stimulate cells (e.g., the cells 140 of FIG. 1) and record excitation of the cells due to the stimulation in the form of an electrical signal. In addition, the electrode array 210 may monitor a state of the cells 140 in real time, and may detect a state change of the cells 140 during the cell culture process. The electrode array 210 may correspond to the MEA 120 of FIG. 1, but embodiments are not limited thereto.

The cells 140 may include neurons such as brain cells or brain neurons, but embodiments are not limited thereto. The cells 140 may generate at least one of an electrical signal, an optical signal, and a chemical signal through neural stimulation by a plurality of electrodes 211 of the electrode array 210. In embodiments, the plurality of electrodes 211 may correspond to the electrodes 130 of FIG. 1, but embodiments are not limited thereto. The cells 140 may be cultured in a cell culture vessel, which may correspond to the cell culture vessel 110 of FIG. 1, but embodiments are not limited thereto.

The electrode array 210 may further include a plurality of AFEs 213, which may correspond to the AFEs 135 of FIG. 1, but embodiments are not limited thereto.

The plurality of electrodes 211 come into contact with the culture medium and/or the cells 140 in order to culture the cells 140.

In embodiments, the plurality of electrodes 211 may be spaced apart from each other at a 20-μm pitch, but embodiments are not limited thereto. The plurality of electrodes 211 may detect or obtain an electrical signal (e.g., a voltage signal or a current signal) generated in a target (e.g., the cells 140) which comes into contact with the plurality of electrodes 211. The cells 140 in contact with each of the plurality of electrodes 211 may generate a voltage signal when the cells 140 are stimulated by an input electrical signal. Each of the plurality of electrodes 211 may detect the voltage signal generated by the target which is in contact with. In embodiments, each input end of the plurality of electrodes 211 may include a switch for a diode connection, examples of which are described in greater detail below.

Each AFE 213 may correspond to a respective electrode 211. For example, when the electrode array 210 includes ten electrodes 211, ten AFEs 213 may also be included in the electrode array 210.

Each AFE 213 may correspond to a component for stimulating the cells 140 coming into contact with the corresponding electrode 211, and for recording the excitation of the cells 140 due to the stimulation in the form of an electrical signal. Each AFE 213 may be, or may include, a block for digitizing an analog signal in a digital system for processing an analog signal into a digital signal. For example, an AFE 213 may be a circuit in which an analog pre-positioning circuit and an analog-digital converter (ADC) are integrated into a single component or chip. Each AFE 213 may include, for example, a stimulator for stimulating the cells 140, a signal amplifier for amplifying a signal detected in the cells 140, and an ADC for converting an amplified (analog) signal into a digital signal, but embodiments are not limited thereto. For example, in some embodiments, each AFE 213 may include, an instrumentation amplifier (IA) and an ADC corresponding to an electrode 211. Some AFEs 213 may amplify an electrical signal measured from corresponding electrodes through a corresponding IAs, and may convert the amplified electrical signals into digital signals through a corresponding ADCs.

For example, each AFE 213 may generate a digital signal based on analog electrical signals received from a corresponding electrode 211 of the electrode array 210, and a reference signal received from a reference electrode of the electrode array 210.

The verification device 200 may determine whether an electrical signal path (e.g., a current path or an impedance path) is generated by connection verifying circuits (CVCs) (e.g., first CVCs 231 and/or a second CVC 233) based on outputs of the plurality of AFEs 213.

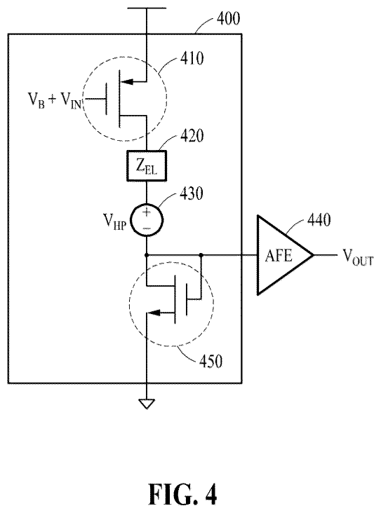

The plurality of AFEs 213 may include, for example, a direct-conversion AFE, which may for example refer to an AFE 213 which includes a direct-conversion ADC. The plurality of AFEs 213 may use a direct-conversion ADC to perform analog-to-digital conversion within a limited area in order to allow the verification device 200 to monitor a large number of areas and channels. As described in greater detail with reference to FIG. 4 below, the verification device 200 according to an embodiment use of the direct-conversion ADC may be advantageous because it may be utilized for any type of voltage readout circuit added to an AFE 213. In embodiments, the verification device 200 may achieve miniaturization of a circuit using the direct-conversion AFE.

In some embodiments, each of the plurality of AFEs 213 may include a first p-channel metal-oxide-semiconductor (PMOS) transistor that operates as a current source. For example, the control circuit 230 may verify second connections between the plurality of electrodes 211 based on whether an electrical signal path is formed between first PMOS transistors included in each of the plurality of AFEs 213 connected to each of the plurality of first CVCs 231.

The control circuit 230 may include a plurality of first CVCs 231 corresponding to the plurality of electrodes 211. In embodiments, each first CVC 231 may correspond to a respective electrode 211. Accordingly, the first CVCs 231 may be referred to as local CVCs.

The control circuit 230 may verify at least one connection from among a plurality of first connections between cells and the plurality of electrodes 211, and a plurality of second connections between the plurality of electrodes 211 of the electrode array 210 according to a result of verifying the first CVCs 231.

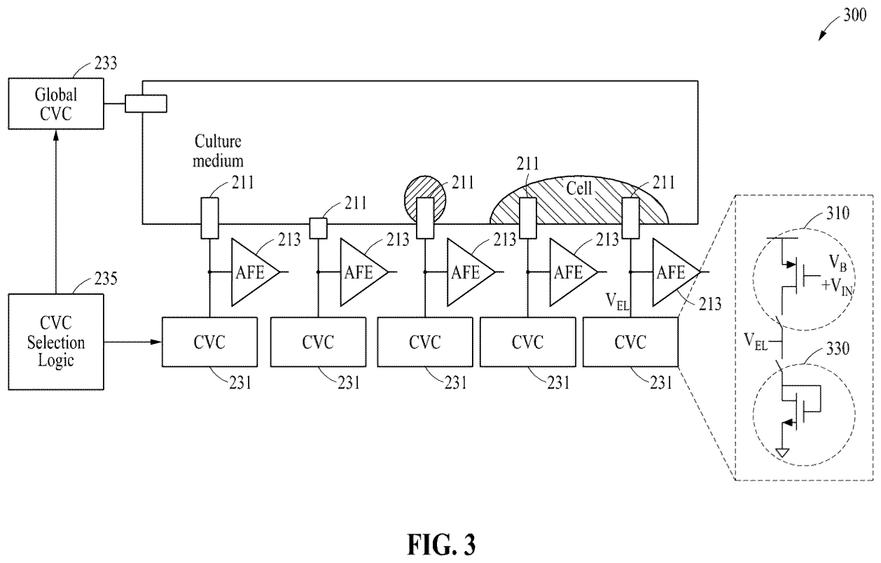

Each of the first CVCs 231 may include a first PMOS transistor (e.g., a first PMOS transistor 310 of FIG. 3 described below) and a first N-channel metal-oxide-semiconductor (NMOS) transistor (e.g., a first NMOS transistor 330 of FIG. 3 described below). The first PMOS transistor may be connected to a first end of a first adjacent electrode that is adjacent to a target electrode that is connected to a target cell from among the plurality of electrodes 211, and may operate as a current source for the first adjacent electrode. The first NMOS transistor may be connected to an input end of an adjacent AFE connected to a second adjacent electrode that is adjacent to the target electrode, and may form an electrical signal path with the first PMOS transistor. The electrical signal path may be, for example, a direct current (DC) current path or an impedance path. The first NMOS transistor may include, for example, a diode-connected NMOS transistor. For example, the diode-connected NMOS transistor may be an NMOS transistor for connecting a drain terminal and a gate terminal of an N-MOSFET transistor to each other such that the corresponding N-MOSFET transistor may constantly operate in a saturation area. When the first NMOS transistor includes the diode-connected NMOS transistor, a current-voltage (I-V) curve may be similar to an I-V curve of a diode device.

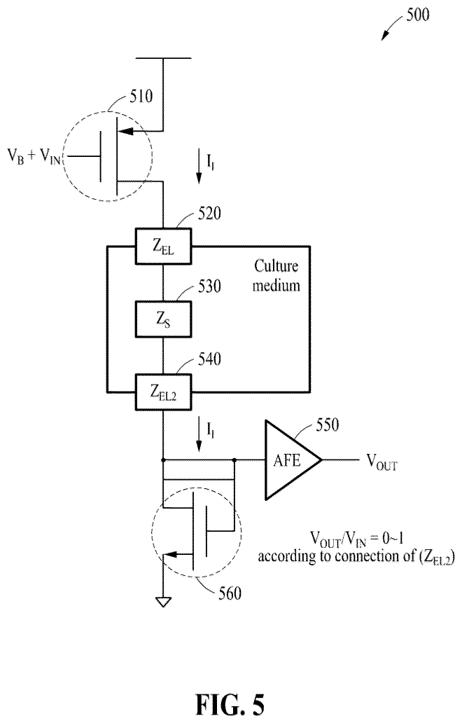

The control circuit 230 may verify a first connection between the target cell and the second adjacent electrode based on a gain of the adjacent AFE connected to the second adjacent electrode. In embodiments, a second connection may correspond to the electrical signal path formed with the AFE along a culture medium-cell-electrode path. An example of a method of verifying the first connection by the control circuit 230 is described in greater detail with reference to FIG. 5 below.

The control circuit 230 may verify the second connections based on whether the electrical signal path is formed between the target electrode and adjacent electrodes (e.g., the first adjacent electrode and the second adjacent electrode) of the plurality of electrodes 211. The second connections may correspond to an electrical signal path formed with the AFE along an electrode-cell-electrode path.

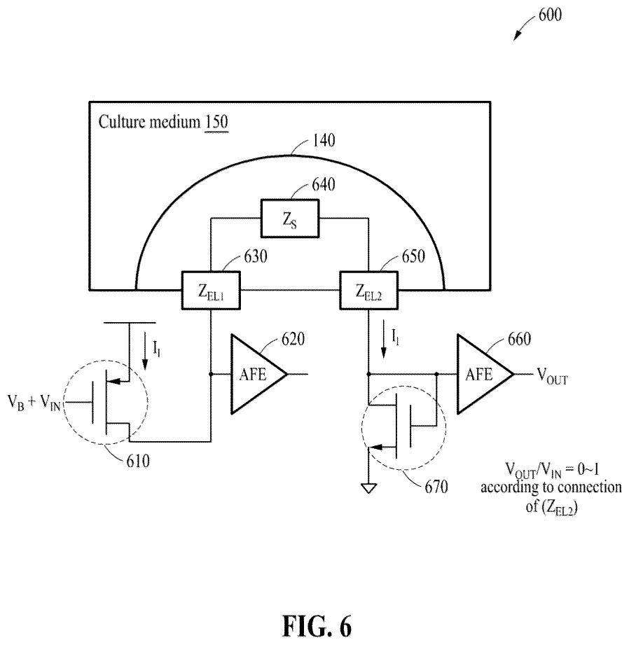

The control circuit 230 may form a current path by connecting a single PMOS transistor (e.g., the first PMOS transistor) and a single NMOS transistor (e.g., the first NMOS transistor) to both ends of each of the plurality of electrodes 211. When the impedance is changed according to the state of the plurality of electrodes 211, the control circuit 230 may verify the second connections between the plurality of electrodes 211 based on changes in a current transferred to the single NMOS transistor and changes in a voltage of a drain terminal of the single NMOS transistor caused by the changed impedance. An example of a method of verifying the second connection between the electrodes using the control circuit 230 is described in greater detail with reference to FIG. 6 below.

The control circuit 230 may determine a degree of capacitive coupling of the adjacent AFE corresponding to the second adjacent electrode by sweeping a reference signal applied to a gate terminal of the first PMOS transistor of each of the first CVCs 231. The reference signal may refer to, for example, a signal having a predetermined voltage, current, amplitude, and/or frequency. The reference signal may be, for example, a sine wave signal, but embodiments are not limited thereto.

The control circuit 230 may further include at least one of the second CVC 233 and a selection logic circuit 235. In some embodiments, the selection logic circuit 235 may be not included in the control circuit 230, or may be not included in the verification device 200.

The control circuit 230 may further include the second CVC 233 including a second PMOS transistor that operates as a current source for the culture medium, and a second NMOS transistor for forming a current path with the second PMOS transistor. The second CVC 233 may supply a current to the entire culture medium. Accordingly, the second CVC may be referred to as a global CVC. The second CVC 233 may include only the second PMOS transistor and may not include the second NMOS transistor.

The selection logic circuit 235 may activate a first target CVC that is connected to at least one target cell corresponding to a verification target among the plurality of first CVCs 231 based on a selection signal of a user. For example, the selection signal of the user may be a signal for selecting cells to be stimulated to observe the excitation of neurons among the plurality of cells, and/or cells with the excitation of neurons that is to be recorded in the form of an electrical signal. The selection signal of the user may be input, for example, as coordinates or indices of the electrode array, or may be input by setting positions of cells on a map provided on a screen through a user interface, but embodiments are not limited thereto.

The verification device 200 may verify that the process of generating the electrodes 211 in the electrode array 210 and culturing the cells was successfully performed by electrically confirming the quality of the connections through the electrode (e.g., the “target electrode”) corresponding to the target cell, and may verify whether the electrode array 210 operates normally at the same time.

The verification device 200 may be used for various applications of the electrode array 210, for example performing neural recording, digital physiology, and/or drug screening. For example, the verification device 200 may include a drug screening device.

In embodiments, drug screening may refer to a process of evaluating pharmacological activity and toxicity of synthetic compounds or natural materials that may become new drug candidates during a drug development process. Drug screening may be broadly divided into molecule-based screening and cell-based screening. Molecule-based screening may refer to screening used to verify efficacy of a drug against a target through direct and/or indirect molecular reactions, and may be performed to measure the efficacy of millions of drugs in a short time in a cost-effective manner. Cell-based screening may be performed by observing a drug-target reaction based on cells due to the target of the drug existing in the cell. In an embodiment, drug screening may refer to cell-based screening.

In drug screening, for example, various drug targets and reactions in the cell may be observed simultaneously using high-content screening (HCS). Using HCS, complex reactions in the cell occurring due to the drug may be observed simultaneously through cell image analysis at multiple wavelengths. Because components in the cell may be connected to a complex network, the cells exposed to the drug may show a complex reaction. The cellular reaction against the drug may cause, not only an on-target effect that is a desired effect of the drug, but also an off-target effect. The off-target effect may correspond to unintended reactions occurring in the cells due to the drug. The verification device 200 according to an embodiment may perform the cell-based HCS to simultaneously detect the on-target effect and the off-target effect of the drug.

FIG. 3 is a diagram illustrating a configuration of a verification device according to an embodiment. FIG. 3 shows a diagram of a configuration of a verification device 300 according to an embodiment.

The verification device 300 may include an input end of each of the plurality of AFEs 213 included in an MEA (e.g., the electrode array 210 of FIG. 2), and the first CVCs 231 on the outside of the electrode array 210. In this case, each of the first CVCs 231 may include one first PMOS transistor 310 and one first NMOS transistor 330.

The first PMOS transistor 310 may be connected to a first end of a first adjacent electrode that is adjacent to a target electrode connected to a target cell among the plurality of electrodes 211 and may operate as a current source for the first adjacent electrode. The first NMOS transistor 330 may be connected to an input end of an adjacent AFE connected to a second adjacent electrode that is adjacent to the target electrode and may form an electrical signal path with the first PMOS transistor 310.

The verification device 300 may determine whether a DC current path is generated from an arbitrary node (e.g., an electrode 211 or an AFE 213) to another node in the electrode array 210 by forming the first CVCs 231 as described above.

For example, an input of each of the first CVCs 231 may be a voltage/current (V/I) conversion through a gate terminal of the first PMOS transistor 310, and an output of each of the first CVCs 231 may be a current/voltage (I/V) conversion through the diode-connected NMOS transistor 330. For example, based on testing connectivity of 1,000 electrodes, there may be vulnerability of process, voltage, temperature (PVT) variation, but such vulnerability may be compensated by adjusting a gain of the AFE.

The verification device 300 may verify the electrode integrity of the plurality of AFEs 213 by enabling a second PMOS transistor of the second CVC 233, and then sequentially enabling the first NMOS transistors of the first CVCs 231 respectively connected to the plurality of AFEs 213 one by one to confirm whether the electrical signal path is formed in each of the first CVCs 231.

For example, the verification device 300 may verify the connection between the plurality of AFEs 213 and the plurality of electrodes 211 by confirming whether the electrical signal path is formed in each of the first CVCs 231 through first activation of the second PMOS transistor of the second CVC 233 and sequential second activations of the first NMOS transistor included in each of the first CVCs 231. If an electrode 211 of the electrode array 210 is not formed properly, or if bubbles are formed in the electrode 322, the electrical signal path between the cell and the electrode 211 may not be formed, and a signal output from the verification device 300 may not be observed.

In addition, when it is desired to verify the connection between a particular AFE 213 and a neighboring adjacent AFE 213 after the cell culture is performed, the verification device 300 may confirm whether the electrical signal path is formed by enabling a PMOS transistor of the particular AFE 213 and an NMOS transistor of the adjacent AFE 213. For example, the verification device 300 may verify the connection between a target AFE 213 and an adjacent AFE 213 by activating the first PMOS transistor 310 of the first CVC 231 connected to the target AFE 213 among the plurality of AFEs 213, activating the first NMOS transistor 330 of the first CVC 231 connected to the adjacent AFE 213 neighboring the target AFE 213 to confirm whether the electrical signal path is formed between a target cell corresponding to the target AFE 213 and a neighboring cell corresponding to the adjacent AFE 213. For example, when two electrodes 211 are connected to a single cell, a test signal or an input signal may be observed at an output end of the verification device 300. As another example, when two electrodes 211 are connected to two cells, the electrical signal path may be disrupted due to impedance of a cell membrane, making it impossible to observe the test signal.

In addition, the verification device 300 may select target cells including cells to be stimulated to observe the excitation of neurons corresponding to a selection signal of the user among the plurality of cells and/or cells with the excitation of neurons that is to be recorded in the form of an electrical signal through the selection logic circuit 235. The verification device 300 may determine whether the DC current path is generated from an arbitrary node to another node in the electrode array 210 by activating the first target CVC 231 connected to the selected target cells.

The verification device 300 may verify intracellular recording through current stimulation, generation of a current path, and generation of a current path between electrodes at one time, thereby verifying the entire process of fabrication of a chip including an MEA, post-processing, cell culture, and the like more efficiently on the chip by itself. The post-processing herein may include various tasks that may be performed after the chip fabrication, for example, filtering of a neural signal, extraction of signal features, and/or analysis of dynamics of neural network connection.

FIG. 4 is a diagram illustrating a structure of a first CVC according to an embodiment. Referring to FIG. 4, a first CVC 400 according to an embodiment may include a single PMOS transistor 410 that operates as a current source, a target electrode 420 connected to a target cell among a plurality of electrodes, and a single NMOS transistor 450 connected to an input end of an AFE 440. In embodiments, the first CVC 400 may correspond to the first CVC 231, the target electrode may correspond to the electrode 211, the AFE 440 may correspond to the AFE 213, the PMOS transistor may correspond to the first PMOS transistor 310, and the NMOS transistor 450 may correspond to the first NMOS transistor 330, but embodiments are not limited thereto. In embodiments, the NMOS transistor 450 may correspond to the diode-connected NMOS transistor described above.

For example, a pseudo voltage clamp circuit may be used to detect the interface conditions described above with reference to the MEA 120. The pseudo voltage clamp circuit may determine a charge generated in a cell membrane by measuring a current after fixing a voltage, and may also determine contact impedance by measuring a current supplied from an external voltage source. However, the method of using the pseudo voltage clamp circuit may be implemented only if a structure for configuring an operational amplifier is provided, and therefore, it may be difficult to use the direct-conversion ADC.

In an embodiment, the direct-conversion ADC may be used by measuring the contact impedance using any type of voltage readout circuit added to an output end of the AFE 440.

In addition, when an electrode comes into contact with a biomedical subject, a half-cell potential V_{HP} may be formed due to a chemical reaction at an interface, and this may be modeled as a DC voltage source 430. When the pseudo voltage clamp circuit is used, the half-cell potential V_{HP} may affect an output to occur an impedance measurement error. In an embodiment, because the amount of current is fixed regardless of V_{HP}, the output may not be affected by V_{HP} according to Equation 1 below:

$${V}_{OUT} = \frac{\text{?}}{\text{?}} {V}_{IN} \qquad Equation 1$$

$$\text{?} \text{indicates text missing or illegible when filed}$$

In Equation 1 above, g_{m,p} may denote trans-conductance for amplification of the single PMOS transistor 410, and g_{m,n} may denote trans-conductance for amplification of the single NMOS transistor 450.

The first CVC 400 may form a current path by connecting the single PMOS transistor 410 and the single NMOS transistor 450 to respective ends of the target electrode 420. The first CVC 400 may verify the connection of the target electrode 420 based on changes in a current transferred to the single NMOS transistor 410 changes, and changes in a drain voltage of the single NMOS transistor 450 caused by impedance changes according to the state of the target electrode 420.

FIG. 5 is a diagram illustrating a method of verifying a first connection between an electrode of an MEA and a cell according to an embodiment. FIG. 5 shows a structure of a control circuit 500 for verifying a first connection between a target electrode coming into contact with a target cell in an MEA according to an embodiment and a culture medium, for example between the target electrode and the target cell included in the culture medium. In embodiments, the control circuit 500 may correspond to the first CVC 231 or the second CVC 233, but embodiments are not limited thereto.

The control circuit 500 may include a PMOS transistor 510 (e.g., the second PMOS transistor included in the second CVC 233) that operates as a current source at the top of the culture medium. In embodiments, at least one of an input signal V_{IN} and a reference signal V_{B} having a predetermined frequency and amplitude may be applied to a gate terminal of the PMOS transistor 510. In addition, a diode-connected NMOS transistor 560 may be connected to an input end of an AFE 550 using a switch.

For example, when an impedance path including Z_{EL1} 520-Z_{S} 530-Z_{EL2} 540 is formed in the control circuit 500, a current I₁ supplied by the current source (e.g., the PMOS transistor 510) may be fully supplied such that a gain of the AFE 550 may be one (“1”). For example, Z_{EL1} 520 may denote a first impedance corresponding to a first adjacent electrode that is adjacent to one side of the target electrode. Z_{S} 530 may denote a second impedance corresponding to the target electrode connected to the target cell. Z_{EL2} 540 may denote a third impedance corresponding to a second adjacent electrode adjacent to the other side of the target electrode.

As another, when the impedance path including Z_{EL1} 520-Z_{S} 530-Z_{EL2} 540 is not properly formed, a portion of the impedance path may be open such that the current source (e.g., the PMOS transistor 510) falls into a triode region, and a gain drop in that the gain of the AFE 550 becomes smaller than one (“1”) may occur. The triode region may correspond to a small unsaturated region of a drain voltage V_{D} in a saturated region where a drain current I_{D} is saturated and a characteristic curve becomes a horizontal line, that is, a region where the drain current I_{D} changes together with the drain voltage V_{D}, in a current-voltage characteristic (I_{D}-V_{Ds} characteristic) between the drain current I_{D} and the drain voltage V_{D} of an MOSFET transistor.

The control circuit 500 may verify the first connection between the target cell and the second adjacent electrode based on the gain of the adjacent AFE 550 connected to the second adjacent electrode.

In addition, the control circuit 500 may verify first connections between the cells and the plurality of electrodes according to the determination whether the gain of the AFE 550 has the value of one (“1”). For example, a value of V_{OUT}/V_{IN} may be between zero (“0”) and one (“1”) based on the first connection between the cell immersed in the culture medium and the electrode.

The control circuit 500 may determine whether a DC current (e.g., the current I₁) is formed by the method described above, and may determine a degree of capacitive coupling of the adjacent AFE 550 corresponding to the second adjacent electrode by sweeping a frequency of the known reference signal V_{B} applied to the gate terminal of the PMOS transistor 510.

FIG. 6 is a diagram illustrating a method of verifying a second connection between electrodes of an MEA according to an embodiment. FIG. 6 illustrates a structure of a control circuit 600 for verifying a connection between the cells 140, for example a second connection between electrodes in contact with the cells 140 according to an embodiment.

The control circuit 600 may verify the second connection between a first adjacent electrode, a target electrode, and a second adjacent electrode based on the determination whether an impedance path or a DC current path is formed by a first impedance Z_{EL1} 630 corresponding to the first adjacent electrode, a second impedance Z_{S} 640 corresponding to the target electrode, and a third impedance Z_{EL2} 650 corresponding to the second adjacent electrode.

For example, when each of a plurality of AFEs 620 and 660 includes a first PMOS transistor that operates as a current source, the control circuit 600 may determine whether the DC current path or the impedance path connecting the first impedance Z_{EL1} 630, the second impedance Z_{S} 640, and the third impedance Z_{EL2} 650 is formed as described above with reference to FIG. 5, thereby determining the connection between the electrodes.

When the impedance connection between electrodes is measured, an AFE from which a current flow starts may be embedded with a PMOS transistor. In FIG. 6, the AFE 620 that operates as a current source may be embedded with the PMOS transistor. For example, the current flow starting from a PMOS transistor 610 may flow through the first impedance Z_{EL1} 630, the second impedance Z_{S} 640, the third impedance Z_{EL2} 650 to an NMOS transistor 670. However, in another example of measuring the impedance connection between the electrodes, when the AFE 660 operates as a current source, the AFE 660 may also be embedded with the PMOS transistor. According to an embodiment, when all of the AFEs are used as a current source to determine the impedance connection between the electrodes, all of the AFEs may be embedded with the PMOS transistors.

In this configuration, because the formation of the DC current path may be determined by an AC-coupled amplifier, the AFE that is generally manufactured in the AC-coupled form may be reused by adding two transistors of the single PMOS transistor 610 and the single NMOS transistor 670, without an additional circuit.

The control circuit 600 may verify the connection between the AFE 620 and the adjacent AFE 660 by activating the first PMOS transistor 610 of a CVC connected to the AFE 620 among the plurality of AFEs, and activating the first NMOS transistor 670 of a CVC connected to the adjacent AFE 660 neighboring the AFE 620 to determine the formation of an electrical signal path.

For example, the reference signal V_{B} may be input to a gate terminal of the first PMOS transistor 610 of a first CVC connected to the AFE 620. Accordingly, the control circuit 600 may verify the second connection by measuring how much attenuation of the reference signal has occurred at the second adjacent electrode using an output value of the adjacent AFE 660 through the second adjacent electrode corresponding to the third impedance Z_{EL2} 650.

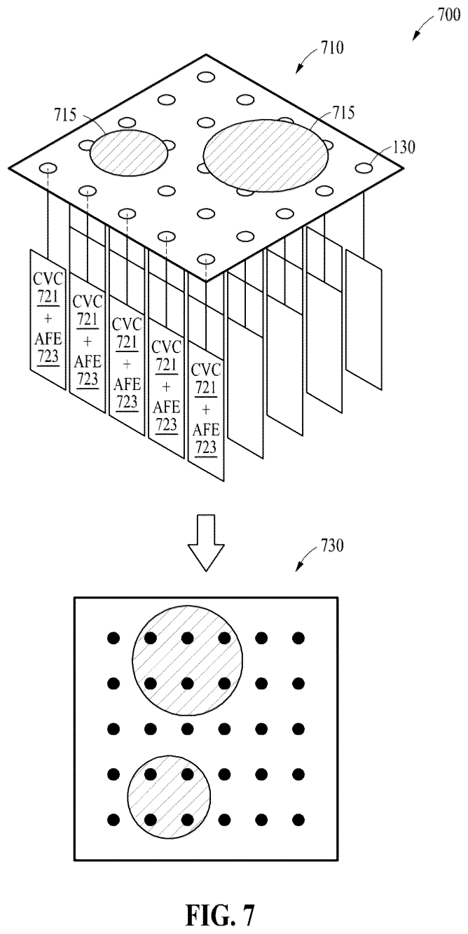

FIG. 7 is a diagram illustrating a method of performing cross verification between a distribution of a target cell calculated by a control circuit and a cell image according to an embodiment. FIG. 7 shows a diagram 710 illustrating an MEA mounted with a plurality of first CVCs 721 (e.g., the first CVCs 231 of FIG. 2) and a plurality of AFEs 723 (e.g., the plurality of AFEs 213 of FIG. 2) and including the plurality of electrodes 130 coming into contact with a plurality of cells, and a result of determining a connection between the plurality of electrodes 130 and the plurality of cells through the first CVCs 721, for example as shown in diagram 730.

A verification device may calculate a distribution of at least one target cell 715 by activating an AFE 723 and a first target CVC 721 connected to at least one target cell 715 corresponding to a verification target as shown in the diagram 710 through a selection logic circuit (e.g., the selection logic circuit 235 of FIG. 2). The verification device may obtain a diagram showing the distribution of the target cell as shown in a diagram 730 through impedance measurement between target electrodes corresponding to the calculated distribution of the at least one target cell 715. The verification device may determine the diagram of the distribution for cells that are much larger than the pitch of each channel in the MEA with reference to FIG. 7.

The verification device may enable more accurate determination of electrical connection by performing cross verification of the electrical connection between the electrode and the cell of the MEA using the drawing of the distribution of the target cells determined through the impedance measurement.

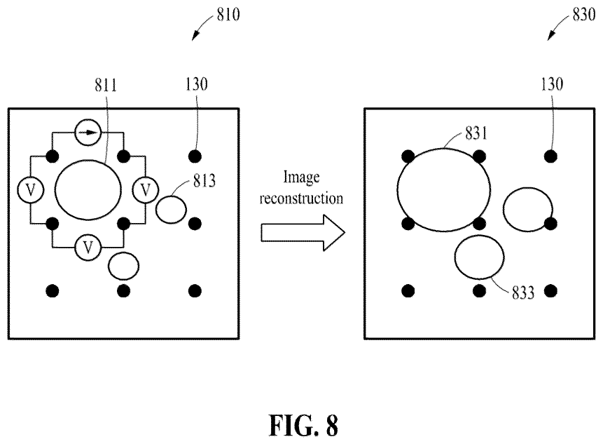

FIG. 8 is a diagram illustrating a method of determining the presence of a small cell that is smaller than a target cell by a control circuit according to an embodiment.

FIG. 8 shows a process of determining the presence of a small cell 833 as shown in a diagram 830 by performing electrical impedance tomography (EIT) when a cell smaller than a pitch of each channel of an MEA according to an embodiment is present as shown in a diagram 810.

For example, when a current path changes due to a cell membrane of a target cell 811 with impedance greater than that of a surrounding cell 813 when applying a current through a current source, a magnitude of a voltage V applied between the electrodes 130 adjacent to the target cell 811 may be changed. For example, a verification device (or a control circuit) may determine whether the small cell 833 smaller than the target cell 831 is present as shown in the diagram 830 by performing image reconstruction based on the magnitude of the voltage changed. For example, the verification device may perform the EIT as shown in the diagram 830 by using a current source assigned to each channel of the MEA and the voltage readout. The verification device may improve capability of analyzing cell dynamics by applying the EIT to the MEA. The verification device may roughly determine a diagram of the distribution of cells smaller than a pitch of each channel of the MEA through the EIT as shown in FIG. 8.

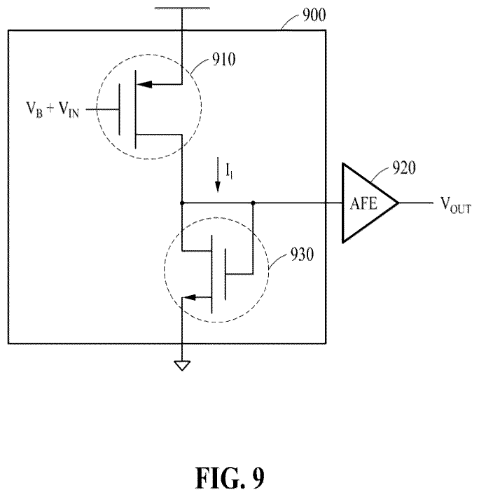

FIG. 9 is a diagram illustrating a method of verifying an integrity of a single channel of a first CVC according to an embodiment. FIG. 9 shows a diagram illustrating a configuration of a CVC 900 for verifying integrity of a channel of an MEA according to an embodiment. The CVC 900 may correspond, for example, the configuration of the CVC 900 for verifying the integrity of the channel itself of the MEA at a stage before the fabrication of the electrode of the MEA. In embodiments, the CVC 900 may correspond to the first CVCs 231 or the CVCs 721 discussed above, but embodiments are not limited thereto.

The control circuit may verify an operation between an AFE 920 and a first PMOS transistor 910 by connecting a first PMOS transistor 910 that operates as a current source in the CVC 900 and a first NMOS transistor 930 that forms an electrical signal path directly to the AFE 920 without a culture medium including cells.

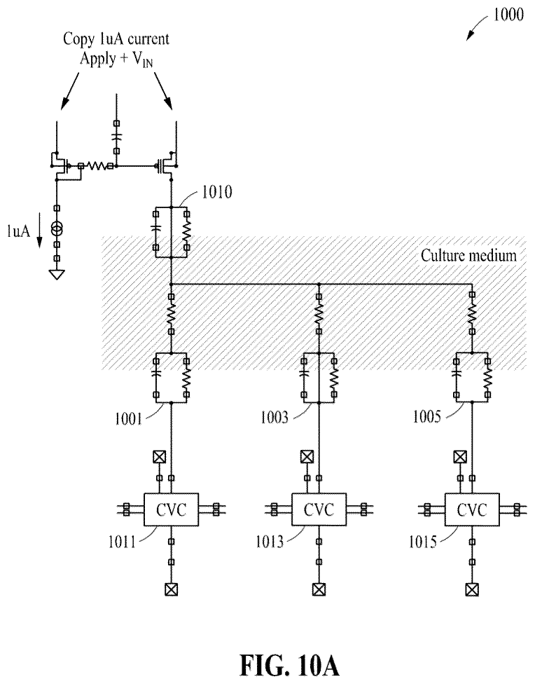

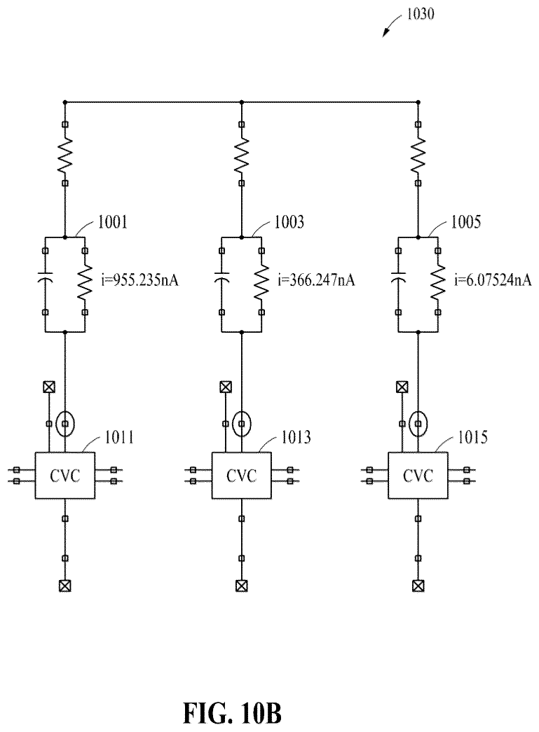

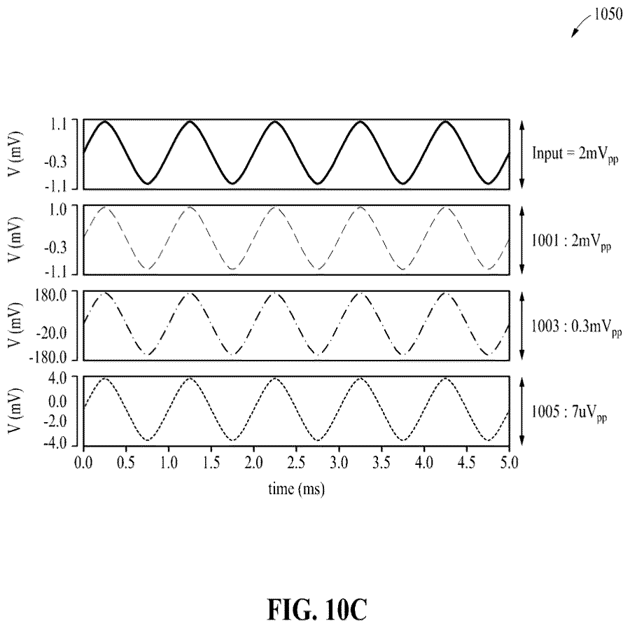

FIG. 10A is a diagram illustrating a simulation circuit for verifying connection between a culture medium and an electrode of an MEA according to an embodiment. FIG. 10A shows a simulation circuit 1000 for verifying a connection between a culture medium and an electrode 1001, an electrode 1003, and an electrode 1005 of the MEA. Here, the MEA may be, for example, a 32 um×32 um MEA on which platinum boride (PtB) is deposited, but embodiments are not limited thereto.

In the simulation circuit 1000 of FIG. 10A, impedances corresponding to three difference example electrodes may be modeled. For example, the electrode 1001 may represent an electrode that operates normally, the electrode 1003 may represent an electrode that experienced a problem during the fabrication process (e.g., corresponding to interface condition 160 of FIG. 1), and the electrode 1005 may represent an electrode on which a bubble is generated (e.g., corresponding to interface condition 170 of FIG. 1). In this case, the electrode 1001 may be modeled with impedance of 100 kOhm/10 nF, the electrode 1003 may be modeled with impedance of 1 MOhm/1 nF, and the electrode 1005 may be modeled with impedance of 100 MOhm/10 nF.

When the simulation circuit 1000 of FIG. 10A applies a current corresponding to a test signal, for example a current of 1 uA, to a culture medium, and enables CVCs 1011, 1013, and 1015 respectively connected to the electrodes 1001, 1003, and 1005, the simulation circuit 1000 may simulate whether an electrical signal path is formed in each of the electrodes 1001, 1003, and 1005. Examples of simulation results of the simulation circuit 1000 are described below with reference to FIGS. 10B and 10C.

FIG. 10B is a diagram illustrating an amount of current applied to the simulation circuit of FIG. 10A that is input to each CVC through electrodes.

FIG. 10B shows a diagram 1030 illustrating an amount of current input to each CVC through the electrode 1001, the electrode 1003, and the electrode 1005 when the current of 1 uA corresponding to the test signal is applied to the culture medium in the simulation circuit 1000 of FIG. 10A.

For the electrode 1001, it may be seen that most of the current (e.g., about 955 nA) of the current of 1 uA applied to the culture medium is injected to the CVC 1011.

In contrast, in a case of the electrode 1003, it may be seen that a portion of current (e.g., about 366 nA) of the current of 1 uA is injected to the CVC 1013, and in the case of the electrode 1005, an insignificant amount of current (e.g., about 6 nA) of the current of 1 uA is injected to the CVC 1015.

As described above, it may be confirmed that the amount of current passing through each of the electrodes 1001, 1003, and 1005 may vary according to the state of each of the electrodes 1001, 1003, and 1005, and the connection between the culture medium and the electrodes 1001, 1003, and 1005 may be verified through the amount of current that may vary.

FIG. 10C is a diagram illustrating a difference between amplitudes of signals according to the amount of current for each electrode of FIG. 10B. Referring to FIG. 10C, the amount of current injected to the CVC varies according to the state of the modeled electrodes 1001, 1003, and 1005 according to an embodiment, and FIG. 10C shows a graph 1050 showing a difference between amplitudes of signals generated in each electrode due to the amount of current that may vary.

For example, when an amplitude of an input test signal is set to 2 mVpp, an amplitude of a signal in the electrode 1001 is 2 mVpp, which is the same as the amplitude of the test signal, but an amplitude of a signal in the electrode 1003 is 0.3 mVpp, and an amplitude of a signal in the electrode 1005 is 7 uVpp. Accordingly, a great difference between amplitudes may be confirmed. Therefore, it may be understood that the state of the electrode may be verified through the amplitude of the signal corresponding to each electrode.

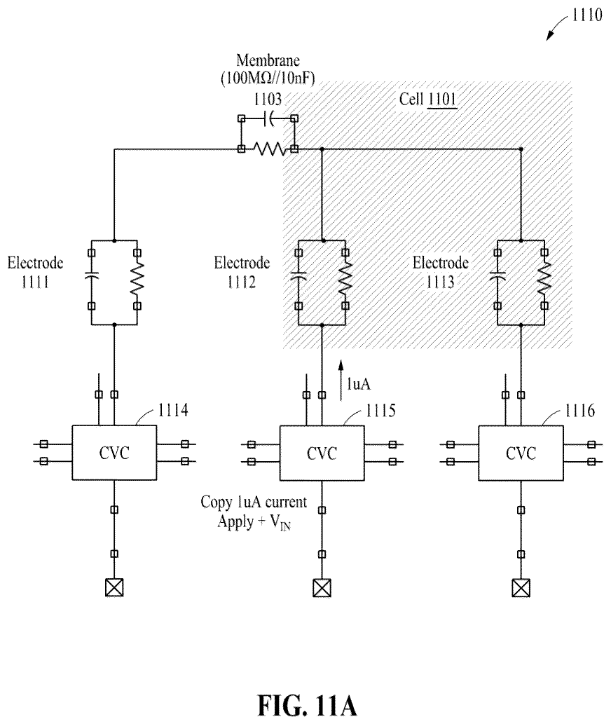

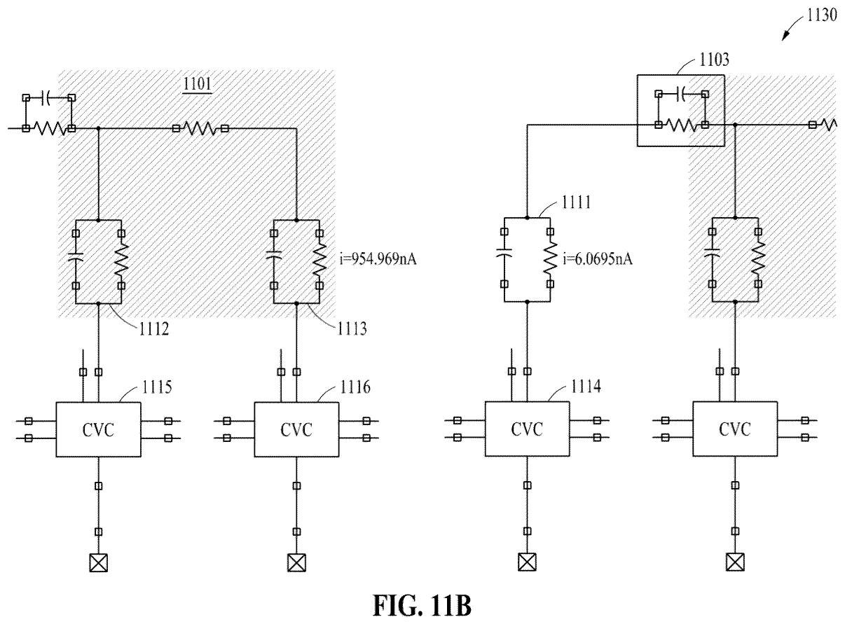

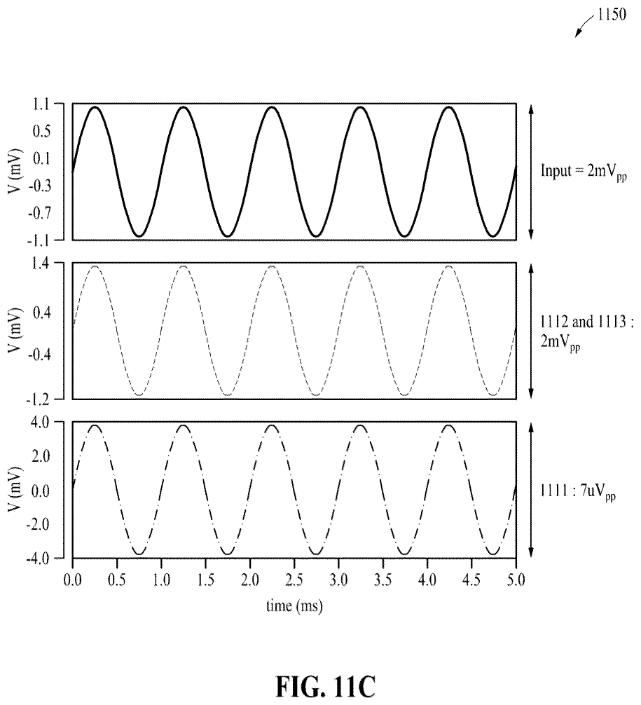

FIG. 11A is a diagram illustrating a simulation circuit for verifying connection between electrodes of an MEA according to an embodiment. FIG. 11A shows a structure of a simulation circuit 1110 that simulates electrical connection through a cell 1101 between AFEs corresponding to an electrode 1111, an electrode 1112, and an electrode 1113 by reflecting impedance information of the inside of the cells 1101 and a cell membrane 1103. For example, the AFEs may be connected to a CVC1114 corresponding to the electrode 1111, a CVC 1115 corresponding to the electrodes 1112, and a CVC 1116 corresponding to the electrode 1113. An impedance of the inside the cell 1101 may be modeled as 500 Ohm, and an impedance of the cell membrane 1103 may be modeled as 100 MOhm.

The impedance of the electrodes 1111, 1112, and 1113 in the simulation circuit 1110 of FIG. 11A may be modeled as though electrodes 1112 and 1113 are connected through the cell 1101, and the electrode 1111 is separated by the cell membrane 1103. The impedance of the electrodes 1111, 1112, and 1113 may all be modeled equally as 100 kOhm/10 nF.

The simulation circuit 1110 may apply a current of 1 uA corresponding to a test signal to a culture medium, and confirm whether the current is injected to the CVCs 1114, 1115, and 1116 while sequentially enabling the CVCs 1114, 1115, and 1116. An example of a simulation result of the simulation circuit 1100 of FIG. 11A is described below with reference to FIGS. 11B and 11C.

FIG. 11B is a diagram illustrating an amount of current applied to the simulation circuit of FIG. 11A that is input to each CVC through the cell connected to the electrode and the cell membrane.

FIG. 11B shows a diagram 1130 illustrating an amount of current input to the corresponding CVCs 1114, 1115, and 1116 through the electrodes 1112 and 1113 connected to the cell 1101 and the electrode 1111 separated by the cell membrane 1103, when the simulation circuit 1110 of FIG. 11A applies the current of 1 uA corresponding to the test signal to the culture medium.

For the electrodes 1112 and 1113 connected to the cell 1101, it may be seen that most of the current (e.g., about 954 nA) of the current 1 uA applied to the culture medium is injected to the CVCs 1115 and 1116.

In contrast, for the electrode 1111 connected to the cell membrane 1103, it may be seen that an insignificant amount of current (e.g., about 6 nA) of the current of 1 uA is injected to the CVC 1114.

As described above, it may be confirmed that the amount of current passing through each electrode may vary depending on a target (e.g., a cell and a cell membrane) connected to the electrode, and the connection between electrodes may be verified through the amount of current that may vary.

FIG. 11C is a diagram illustrating a difference between amplitudes of signals according to the amount of current for each electrode of FIG. 11B. FIG. 11C shows a graph 1150 showing a difference between amplitudes of signals generated in each electrode as the amount of current injected to each CVC varies for each target connected to the electrodes modeled according to an embodiment.

For example, when an amplitude of an input test signal is set to 2 mVpp, an amplitude of a signal in the electrodes 1112 and 1113 connected to the cell 1101 is 2 mVpp which is the same as the amplitude of the test signal, but an amplitude of a signal in the electrode 1111 separated by the cell membrane 1103 is 7 uVpp. Accordingly, a great difference between amplitudes may be confirmed. Therefore, the formation of the electrical signal path may be confirmed through the amplitude of the signal corresponding to each electrode.

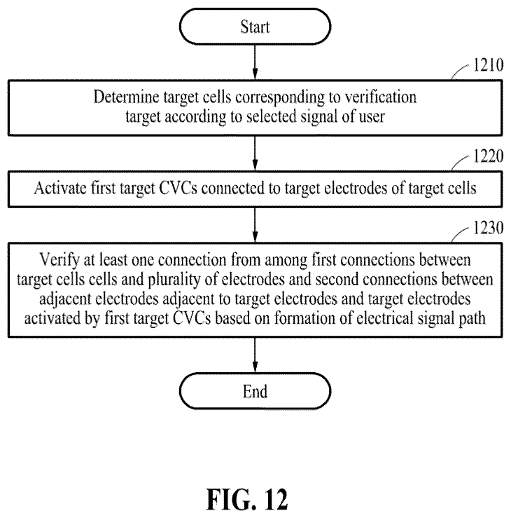

FIG. 12 is a flowchart illustrating a method of operating a verification device according to an embodiment. Operations described hereinafter with reference to FIG. 12 may be performed sequentially, but embodiments are not limited thereto. For example, the order of the operations may be changed and at least two of the operations may be performed in parallel.

Referring to FIG. 12, a verification device according to an embodiment may verify at least one connection through operations 1210 to 1230.

At operation 1210, the verification device determines target cells corresponding to a verification target according to a selected signal of a user.

At operation 1220, the verification device activates a first target CVC connected to target electrodes of the target cells determined in operation 1210.

At operation 1230, the verification device may verify at least one connection from first connections between the target cells and the plurality of electrodes and second connections between adjacent electrodes adjacent to the target electrodes and the target electrodes activated by the first target CVC based on the formation of an electrical signal path. For example, the target electrodes may respectively correspond to the first target CVCs from among the plurality of electrodes in an electrode array.

The embodiments described herein may be implemented using a hardware component, a software component, and/or a combination thereof. A processing device may be implemented using one or more general-purpose or special-purpose computers, such as, for example, a processor, a controller, an arithmetic logic unit (ALU), a digital signal processor (DSP), a microcomputer, a field programmable gate array (FPGA), a programmable logic unit (PLU), a microprocessor or any other device capable of responding to and executing instructions in a defined manner. The processing device may run an operating system (OS) and one or more software applications that run on the OS. The processing device also may access, store, manipulate, process, and generate data in response to execution of the software. For purpose of simplicity, the description of a processing device is used as singular; however, one skilled in the art will appreciate that a processing device may include multiple processing elements and/or multiple types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or uniformly instruct or configure the processing device to operate as desired. Software and data may be stored in any type of machine, component, physical or virtual equipment, or computer storage medium or device capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter.

The above-described hardware devices may be configured to act as one or more software modules in order to perform the operations of the above-described embodiments, or vice versa.

Although some embodiments have been described with reference to the drawings, embodiments are not limited thereto, and the scope of the disclosure may be understood to include ly various technical modifications and variations based thereon. For example, according to embodiments, the processes described above may be performed in a different order, and components in a described system, architecture, device, or circuit may be combined in a different manner and/or replaced or supplemented by other components or their equivalents. Accordingly, other implementations are within the scope of the following claims.

What is claimed is:

1. A verification device comprising:
an electrode array comprising a plurality of electrodes configured to contact a culture medium for culturing a plurality of cells, and a plurality of analog front ends (AFEs) corresponding to the plurality of electrodes; and
a control circuit comprising a plurality of first connection verifying circuits (CVCs) corresponding to the plurality of electrodes,
wherein the control circuit is configured to verify at least one connection from among a plurality of first connections between the plurality of cells and the plurality of electrodes, and a plurality of second connections between the plurality of electrodes based on a result of verifying the plurality of first CVCs.

2. The verification device of claim 1, wherein each first CVC from among the plurality of first CVCs comprises:
a first p-channel metal-oxide-semiconductor (PMOS) transistor connected to a first adjacent electrode that is adjacent to a target electrode connected to a target cell from among the plurality of electrodes, wherein the first PMOS transistor is configured to operate as a current source for the first adjacent electrode; and
a first n-channel metal-oxide-semiconductor (NMOS) transistor connected to an input end of an adjacent AFE connected to a second adjacent electrode that is adjacent to the target electrode, wherein the first NMOS transistor is configured to form an electrical signal path with the first PMOS transistor.

3. The verification device of claim 2, wherein the control circuit is further configured to verify a first connection between the target cell and the second adjacent electrode based on a gain of the adjacent AFE connected to the second adjacent electrode.

4. The verification device of claim 1, wherein the control circuit is further configured to verify a first connection between a cell from among the plurality of cells and an electrode from among the plurality of electrodes by determining whether a gain of an AFE from among the plurality of AFEs corresponding to a first CVC from among the plurality of first CVCs has a first value.

5. The verification device of claim 2, wherein the control circuit is further configured to determine a degree of capacitive coupling of the adjacent AFE corresponding to the second adjacent electrode by sweeping a reference signal applied to a gate terminal of the first PMOS transistor of each first CVC from among the plurality of first CVCs.

6. The verification device of claim 2, wherein the control circuit is further configured to verify a second connection from among the plurality of second connections by determining whether the electrical signal path is formed between the first adjacent electrode, the second adjacent electrode, and the target electrode.

7. The verification device of claim 6, wherein the control circuit is further configured to verify the second connection by determining whether an impedance path is formed by a first impedance corresponding to the first adjacent electrode, a second impedance corresponding to the target electrode, and a third impedance corresponding to the second adjacent electrode.

8. The verification device of claim 1, wherein the plurality of AFEs comprises a direct-conversion AFE.

9. The verification device of claim 1, wherein the first NMOS transistor comprises a diode-connected NMOS transistor.

10. The verification device of claim 1, wherein each AFE of the plurality of AFEs comprises a first p-channel metal oxide semiconductor (PMOS) transistor configured to operate as a current source, and
wherein the control circuit is further configured to verify the plurality of second connections by determining whether an electrical signal path is formed between first PMOS transistors included in AFEs connected to the plurality of first CVCs.

11. The verification device of claim 1, wherein the control circuit further comprises a second CVC comprising a second p-channel metal oxide semiconductor (PMOS) transistor configured to operate as a current source for the culture medium, and a second n-channel metal oxide semiconductor (NMOS) transistor configured to form a current path with the second PMOS transistor.

12. The verification device of claim 11, wherein the control circuit is further configured to verify a connection between the plurality of AFEs and the plurality of electrodes by determining whether an electrical signal path is formed in each first CVC from among the plurality of first CVCs by activating the second PMOS transistor and sequentially activating first NMOS transistors included in the plurality of first CVCs.

13. The verification device of claim 2, wherein the control circuit is further configured to verify a connection between a target AFE and the adjacent AFE by determining whether the electrical signal path is formed by activating the first PMOS transistor of a first CVC connected to a target AFE from among the plurality of AFEs, and activating the first NMOS transistor of a first CVC connected to the adjacent AFE.

14. The verification device of claim 2, wherein the control circuit is further configured to verify an operation between the adjacent AFE and the first PMOS transistor by directly connecting the first PMOS transistor to the first NMOS transistor without the culture medium.

15. The verification device of claim 1, wherein the control circuit further comprises a selection logic circuit configured to activate a first target CVC connected to at least one target cell corresponding to a verification target from among the plurality of first CVCs.

16. The verification device of claim 15, wherein the control circuit is further configured to calculate a distribution of the at least one target cell by activating the first target CVC using the selection logic circuit.

17. The verification device of claim 2, wherein the control circuit is further configured to detect a small cell that is smaller than the target cell by performing image reconstruction based on a magnitude of a voltage applied between electrodes adjacent to the target cell being changed as a current path changes while a current is applied through the current source due to a cell membrane of the target cell having an impedance greater than an impedance of a surrounding cell.

18. The verification device of claim 1, wherein the verification device comprises a drug screening device.

19. A method of operating a verification device, the method comprising:
selecting a plurality of target cells corresponding to a verification target according to a selection signal of a user;
activating a plurality of first target connection verifying circuits (CVCs) connected to a plurality of target electrodes corresponding to the plurality of target cells; and
verifying at least one connection from among a plurality of first connections between the plurality of target cells and the plurality of target electrodes, and a plurality of second connections between the plurality of target electrodes and a plurality of adjacent electrodes that are adjacent to the plurality of target electrodes.

20. A non-transitory computer-readable storage medium storing instructions which, when executed by a processor of a verification device, cause the verification device to:
select a plurality of target cells corresponding to a verification target according to a selection signal of a user;
activate a plurality of first target connection verifying circuits (CVCs) connected to a plurality of target electrodes corresponding to the plurality of target cells; and
verify at least one connection from among a plurality of first connections between the plurality of target cells and the plurality of target electrodes, and a plurality of second connections between the plurality of target electrodes and a plurality of adjacent electrodes that are adjacent to the plurality of target electrodes.
